(19) United States
(12) Patent Application Publication
(10) Pub. No.: US 2010/0247476 A1
(43) Pub. Date: Sep. 30, 2010

(54) Copolymer Containing Water-Soluble Lipids

(75) Inventors: Stephanie Von Thaden, Hamburg (DE); Sophie Viala, Koln (DE); Anja Bauer, Hamburg (DE)

Correspondence Address: AIR LIQUIDE USA LLC;Intellectual Property, 2700 POST OAK BOULEVARD, SUITE 1800, HOUSTON, TX, 77056, US

(73) Assignee: Societe d'Exploitation de Products Pour Les Industries Chimiques SEPPIC, Paris (FR)

(21) Appl. No.: 12/665,951

(22) PCT Filed: Jun. 23, 2008
(86) PCT No.: PCT/EP2008/005037

§ 371 Date: May 19, 2010

(30) Foreign Application Priority Data
Jun. 25, 2007 (DE) 10 2007 029 735.3

(51) Int. Cl. A61K 8/81

(52) U.S. Cl. 424/78.03

(57) ABSTRACT

The invention relates to a cosmetic preparation which contains: a) a hydroxyethylacrylate/sodium acryloyldimethyl taurate copolymer, b) one or two water-soluble lipids

The present invention relates to cosmetic preparations containing a combination of water-soluble and/or water-dispersible lipids and a copolymer of hydroxyethyl acrylate/sodium acryloyldimethyltaurate.

Polymeric compounds, for example polyacrylates or polysaccharides, have been used for many years as “thickeners” for increasing the viscosity in particular of aqueous foods, cosmetic preparations, construction materials, etc.

A particular challenge is the thickening of cosmetic preparations. Cosmetic preparations are sometimes highly complex mixtures (for example aqueous gels or emulsions) of highly diverse chemical compounds. A thickener for cosmetic preparations must therefore have high compatibility towards a large number of substances. Despite the (seemingly) large selection of polymeric thickeners, for the person skilled in the art it has until now regularly been a major problem to find a thickener suitable for a cosmetic preparation. In particular, the thermal stability, storage stability, tolerance towards other ingredients and the cosmetic performance present the person skilled in the art with difficulties during the formulation of a cosmetic preparation.

A particular problem when thickening cosmetic preparations is that the preparations become sticky as a result of adding the polymeric thickener and have a tendency towards “thread drawing”. The increasing stickiness makes the products sensorially unattractive. Stickiness and the tendency toward “thread drawing” moreover hinders removal from the storage vessel. These phenomena arise in a particularly troublesome manner with gel-like preparations (in particular transparent gels) and with preparations which comprise water-soluble and/or water-dispersible lipids (e.g. ethoxylated silicone derivatives).

It was therefore the object of the present invention to overcome the disadvantages of the prior art and to develop a new, sensorially more attractive and easy-to-produce viscous cosmetic product.

Surprisingly, the object is achieved by a cosmetic transparent or translucent preparation comprising
a) hydroxyethyl acrylate/sodium acryloyldimethyl-taurate copolymer,
b) one or more water-soluble and/or water-dispersible lipids.

Although the person skilled in the art knows of EP 1496081, this specification was unable to point the way to the present invention.

It is advantageous according to the invention if the preparation according to the invention comprises hydroxyethyl acrylate/sodium acryloyldimethyltaurate copolymer in a concentration of from 0.1 to 3% by weight, based on the total weight of the preparation.

It is preferred according to the invention if the preparation according to the invention comprises hydroxyethyl acrylate/sodium acryloyldimethyltaurate copolymer in a concentration of from 0.2 to 2.0% by weight, based on the total weight of the preparation.

In this connection, it is preferred according to the invention if the compound with the CAS No.: 111286-86-3 is used as hydroxyethyl acrylate/sodium acryloyl-dimethyltaurate copolymer.

It is advantageous according to the invention if the preparation comprises water-soluble and/or water-dispersible lipids in a total concentration of from 0.01 to 20% by weight, based on the total weight of the preparation.

It is preferred according to the invention if the preparation comprises water-soluble lipids in a total concentration of from 0.05 to 10% by weight, based on the total weight of the preparation.

According to the invention, “water-soluble and/or water-dispersible lipids” are understood as meaning those lipids which are water-soluble or water-dispersible at room temperature (20° C.).

According to the invention, it is advantageous if one or more compounds selected from the group of the compounds ethoxylated (PEG or PPG) silicone oils (such as e.g. dimethicones or methyl ether dimethyl silicones), ethoxylated synthetic or natural esters or quaternized silicone oils are used as water-soluble and/or water-dispersible lipids.

It is preferred according to the invention if one or more compounds selected from the group consisting of ethoxylated derivatives (PEG modification, PPG modification or both) of dimethicone, glycerides, ricinoleates, castor oil, methyl ether dimethyl silanes, olivates, cocoates, methicones, methyl glucose ethers, cocoglycerides, olive glycerides, sunflower glycerides, laurates, distearates, avocadoates, beeswax are used as water-soluble and/or water-dispersible lipids.

According to the invention, it is advantageous if the preparation according to the invention is in the form of a gel, in particular a transparent or translucent gel.

Here, transparent or translucent gels are preferred according to the invention.

According to the invention, it is particularly preferred if the preparation according to the invention has a transmission at 420 nm of 60-100%.

The transmission according to the invention at room temperature is measured here using the Agilent 8453 UV-Visible spectrophotometer.

According to the invention, the preparation according to the invention advantageously has a viscosity of from 100 to 20 000 mPas, preferably from 1000 to 10 000 mPas, measured using the instrument R123 from Rheo (Athengstett, Germany), spindle 1 at 25° C.

According to the invention, it is advantageous if the preparation according to the invention comprises one or more perfume substances selected from the group of the compounds limonene [5989-27-5], citral, linalool [78-70-6], alpha-isomethylionone [1335-46-2], geraniol [106-24-1], citronellol [106-22-9], [24851-98-7], [18479-58-8], [54464-57-2], [80-54-6], [1222-05-5], [32388-55-9], [105-95-3], [31906-04-4], [8008-57-9], [32210-23-4], [120-57-0], [115-95-7], [101-86-0], [140-11-4], [6259-76-3] and [127-51-5], 2-isobutyl-4-hydroxy-4-methyltetrahydropyran, 2-tert-pentylcyclo-hexyl acetate, 3-methyl-5-phenyl-1-pentanol, 7-acetyl-1,1,3,4,4,6-hexamethyltetralin, adipic acid diester, alpha-amylcinnamaldehyde, alpha-methylionone, amyl C, butylphenylmethylpropionalcinnamal, amyl salicylate, amylcinnamyl alcohol, anise alcohol, benzoin, benzyl alcohol, benzyl benzoate, benzyl cinnamate, benzyl salicylate, bergamot oil, bitter orange oil, butyl-phenylmethylpropional, cardamom oil, cedrol, cinnamal, cinnamyl alcohol, citronellyl methylcrotonate, lemon oil, coumarin, diethyl succinate, d-limonenes, ethyllinalool, eugenol, Evernia furfuracea extract, Evernia prunastri extract, farnesol, guaiac wood oil, hexylcinnamal, hexyl salicylate, hydroxycitronellal, hydroxyisohexyl 3-cyclohexenecarboxaldehyde, lavender oil, lemon oil, linalyl acetate, mandarin oil, menthyl PCA, methylheptenone, nutmeg oil, rosemary oil, sweet orange oil, terpineol, tonka bean oil, triethyl citrate and/or vanillin.

In this connection, according to the invention, it is advantageous if the preparation according to the invention comprises one or more perfume substances in a total concentration of from 0.001 to 1% by weight, based on the total weight of the preparation.

Here, it is preferred according to the invention if the preparation according to the invention comprises one or more perfume substances in a total concentration of from 0.001 to 0.5% by weight, based on the total weight of the preparation.

It is advantageous according to the invention if the preparation according to the invention comprises 0.1 to 99.9% by weight of glycerol, based on the total weight of the preparation.

It is preferred according to the invention if the preparation according to the invention comprises from 0.1 to 15% by weight of glycerol, based on the total weight of the preparation.

It is advantageous according to the invention if the preparation according to the invention comprises one or more solubility promoters in a total concentration of from 0.005 to 15% by weight, based on the total weight of the preparation.

It is preferred according to the invention if the preparation according to the invention comprises one or more solubility promoters in a total concentration of from 0.25 to 10% by weight, based on the total weight of the preparation.

According to the invention, one or more compounds from the group of the compounds of the ethoxylated carbohydrates or fatty acid derivatives can advantageously be used as solubility promoter according to the invention, for example polyoxyethylene(20) sorbitan monolaurate (Tween 20) or polyoxyethyleneoxy-propylene monostearate (Atlas G-2162), propylene glycol, caprylyl/capryl glucosides, sodium lauroyl-sarcosinate, C12-13 alkyl lactates.

According to the invention, the preparation according to the invention advantageously comprises ethanol.

According to the invention, ethanol-containing preparations according to the invention advantageously comprise ethanol in a concentration of from 0.001 up to 30% by weight, based on the total weight of the preparation, preferably 0.001 to 10% by weight.

According to the invention, the preparation according to the invention advantageously comprises one or more compounds selected from the group of the compounds isopropanol, propylene glycol, ethylene glycol, ethylene glycol monoethyl or monobutyl ether, propylene glycol monomethyl, monoethyl or monobutyl ether, diethylene glycol monomethyl or monoethyl ether and analogous products, also alcohols of low carbon number, e.g. ethanol, isopropanol, 1,2-propanediol, 2-methyl-1,3-propanediol; 1,2-pentanediol; 1,2-hexanediol, 1,2-octanediol.

It is preferred according to the invention if the preparation comprises one or more diols selected from the group of the compounds 2-methyl-1,3-propanediol, pentane-1,2-diol, hexane-1,2-diol, heptane-1,2-diol, octane-1,2-diol, nonane-1,2-diol, decane-1,2-diol.

It is advantageous according to the invention if the preparation comprises one or more further UV filters selected from the group of the compounds phenylene-1,4-bis(2-benzimidazyl)-3,3′-5,5′-tetrasulfonic acid salts; 2-phenylbenzimidazole-5-sulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl)-sulfonic acid salts; 2,2′-methylenebis(6-(2H-benzo-triazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[(trimethylsilyl)oxy]disiloxan-yl]propyl]phenol; 3-(4-methylbenzylidene)camphor; 3-benzylidenecamphor; ethylhexyl salicylate; terephthalidenedicamphorsulfonic acid; 2-ethylhexyl 4-(dimethylamino)benzoate; amyl 4-(dimethylamino)-benzoate; 2-ethylhexyl 4-methoxybenzalmalonate; 2-ethylhexyl 4-methoxycinnamate; isoamyl 4-methoxy-cinnamate; 2-hydroxy-4-methoxybenzophenone, 2-hydroxy-4-methoxy-4′-methylbenzophenone; 2,2′-dihydroxy-4-methoxybenzophenone; hexyl 2-(4′-diethylamino-2′-hydroxybenzoyl)benzoate, 4-(tert-butyl)-4′-methoxydi-benzoylmethane; homomethyl salicylate; 2-ethylhexyl 2-hydroxybenzoate; 2-ethylhexyl 2-cyano-3,3-diphenyl-acrylate; dimethicodiethyl benzalmalonate; 3-(4-(2,2-bisethoxycarbonylvinyl)phenoxy)propenyl)methoxy-siloxane/dimethylsiloxane copolymer; 2,4-bis{[4-(2-ethylhexyloxy)-2-hydroxy]phenyl}-6-(4-methoxyphenyl)-1,3,5-triazine (INCI: Bis-Ethylhexyloxyphenol Methoxy-phenyl Triazine); dioctylbutylamidotriazone (INCI: Diethylhexyl-Butamidotriazone); 2,4-bis[5-1(dimethyl-propyl)benzoxazol-2-yl(4-phenyl)imino]-6-(2-ethyl-hexyl)imino-1,3,5-triazine with the CAS No. 288254-16-0; tris(2-ethylhexyl) 4,4′,4″-(1,3,5-triazine-2,4,6-triyltriimino)trisbenzoate (also: 2,4,6-tris[aniline(p-carbo-2′-ethyl-1′-hexyloxy)]-1,3,5-triazine (INCI: Ethylhexyl Triazone); titanium dioxide, zinc oxide, merocyanines selected from the group of the compounds

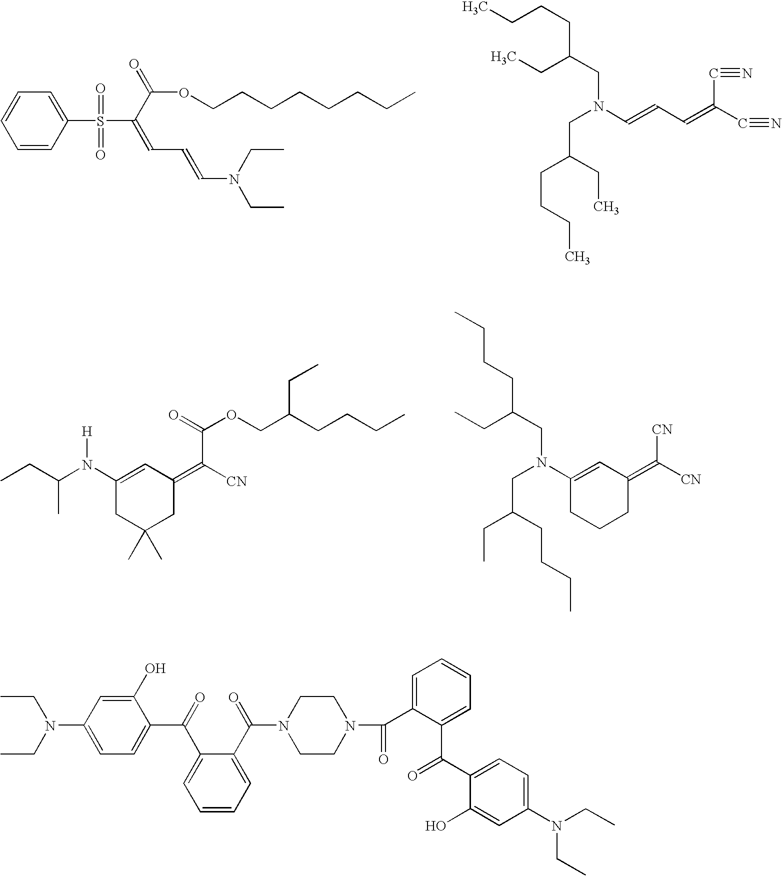

in a concentration of from 0.01 to 40% by weight, based on the total weight of the preparation.

Embodiments advantageous according to the invention of the present invention are characterized in that the preparation comprises, as further ingredients, one or more compounds selected from the group of the compounds alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, tocopheryl acetate, dihydroxyacetone; 8-hexadecene-1,16-dicarboxylic acid, glycerylglucose, (2-hydroxy-ethyl)urea, vitamin E and its derivatives and/or licochalcone A, sodium hyaluronate, dioic acid, salicylic acid.

Active ingredients of this type can advantageously be used in an individual concentration of from 0.01 to 10% by weight, based on the total weight of the preparation.

Furthermore, the preparations according to the invention can advantageously also comprise self-tanning substances, such as, for example, dihydroxyacetone and/or melanine derivatives in concentrations of from 1% by weight to 10% by weight, based on the total weight of the preparation.

In addition, the preparations according to the invention can advantageously also comprise repellents for protecting against flies, ticks and spiders. For example, N,N-diethyl-3-methylbenzamide (trade name: Meta-delphene, “DEET”), dimethyl phthalate (trade name: Palatinol M, DMP), 1-piperidinecarboxylic acid 2-(2-hydroxyethyl)-1-methylpropyl ester and in particular 3-(N-n-butyl-N-acetylamino)propionic acid ethyl ester (available under the trade name Insekt Repellent® 3535 from Merck). The repellents can be used either individually or in combination.

Particularly advantageous preparations are also obtained if antioxidants are used as additives or active ingredients. According to the invention, the preparations advantageously comprise one or more antioxidants. Favorable, but nevertheless optional antioxidants that can be used are all antioxidants customary or suitable for cosmetic applications.

Such antioxidants are advantageously selected from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, chloro-genic acid and derivatives thereof, lipoic acid and derivatives thereof (e.g. dihydrolipoic acid), auro-thioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodi-propionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to pmol/kg), also (metal) chelating agents (e.g. α-hydroxy fatty acids, palmitic acid, phytic acid, phytin, lacto-ferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, tocopherols and derivatives (e.g. vitamin E acetate), vitamin A and derivatives (vitamin A palmitate) and coniferyl benzoate of benzoin resin, rutinic acid and derivatives thereof, butylhydroxytoluene, butylhydroxy-anisole, nordihydroguaiacic acid, nordihydroguaiaretic acid, trihydroxybutyrophenone, uric acid and derivatives thereof, mannose and derivatives thereof, sesamol, sesamolin, zinc and derivatives thereof (e.g. ZnO, ZnSO₄), selenium and derivatives thereof (e.g. selenomethionine) stilbenes and derivatives thereof (e.g. stilbene oxide, trans-stilbene oxide) and the derivatives (salts, esters, ethers, sugars, nucleotides, nucleosides, peptides and lipids) suitable according to the invention of these specified active ingredients.

The amount of the aforementioned antioxidants (one or more compounds) in the preparations is preferably 0.001 to 30% by weight, particularly preferably 0.05-20% by weight, in particular 1-10% by weight, based on the total weight of the preparation.

If vitamin E and/or derivatives thereof are the additional antioxidant or antioxidants, it is advantageous to select their respective concentrations from the range from 0.001-10% by weight, based on the total weight of the formulation.

If vitamin A or vitamin A derivatives, or carotenes or derivatives thereof are the additional antioxidant or antioxidants, it is advantageous to select their particular concentrations from the range from 0.001-10% by weight, based on the total weight of the formulation.

According to the invention, the preparation according to the invention advantageously comprises moisturizers. Advantageous moisturizers within the context of the present invention are, for example, lactic acid and/or lactates, in particular sodium lactate, butylene glycol, propylene glycol, biosaccharide gum-1, glycine soya, ethylhexyloxyglycerol, pyrrolidonecarboxylic acid and urea. In addition, it is particularly advantageous to use polymeric moisturizers from the group of water-soluble and/or water-swellable and/or water-gelable polysaccharides. For example, hyaluronic acid, chitosan and/or a fucose-rich polysaccharide which is listed in the Chemical Abstracts under the registration number 178463-23-5 and is available e.g. under the name Fucogel®1000 from SOLABIA S.A., are particularly advantageous. Moisturizers can advantageously also be used as antiwrinkle active ingredients for protecting against skin changes as occur e.g. during skin ageing.

It is advantageous within the context of the present invention if the preparation according to the invention comprises one or more moisturizers in a total concentration of from 0.1 to 20% by weight and preferably in a total concentration of from 0.5 to 10% by weight, in each case based on the total weight of the preparation.

The cosmetic preparations according to the invention can comprise cosmetic auxiliaries as are customarily used in such preparations, e.g. preservatives, preservative aids, complexing agents, bactericides, substances for preventing or increasing foaming, dyes, pigments which have a coloring effect.

It is advantageous according to the invention if the preparation comprises one or more parabens (e.g. methyl-, ethyl-, propyl-, butylparaben).

### Comparative Experiment

| | 1 | 2 |
|---|---|---|
| PPG-1-PEG-9 lauryl glycol | 0.5 | 0.5 |
| ether | | |
| Glycerol | 2.0 | 2.0 |
| PEG-17 dimethicone | 1.0 | 1.0 |
| Acrylates/C10-30 alkyl | 0.7 | |
| acrylate crosspolymer¹ | | |
| Hydroxyethyl | | 0.7 |
| acrylate/sodium | | |
| acryloyldimethyl taurate | | |
| copolymer² | | |
| Perfume | 0.08 | 0.08 |
| Preservative | 0.6 | 0.6 |
| Sodium hydroxide | q.s., pH = 7 | q.s., pH = 7 |
| Aqua | ad 100 | ad 100 |

¹Carbopol ETD 2020, Noveon
²Sepinov EMT 10, Seppic

0.4 g of a formulation stored at 25° C. is applied between thumb and index finger. The formulation is carefully pressed together and the fingers moved apart. The process is carried out 3 times at a speed of 2 movements/s. The length of the point or the length of the product thread which is formed is assessed.

| | 1 | 2 |
|---|---|---|
| Length of the point in cm | 2.0 | 0.05 |

### EXAMPLES

The examples below are intended to illustrate the present invention without limiting it. The data always refer to % by weight unless stated otherwise.

| | 1 | 2 | 3 | 4 | 5 |
|---|---|---|---|---|---|
| PPG-1-PEG-9 lauryl | 0.5 | 1.0 | 0.5 | 0.7 | 0.6 |
| glycol ether | | | | | |
| Glycerol | 38.5 | 2.0 | 8.0 | 5.0 | 2.0 |
| Hydroxyethyl acrylate/ | 1.0 | 0.8 | 0.5 | 0.2 | 2.0 |
| sodium | | | | | |
| acryloyldimethyltaurate | | | | | |
| copolymer | | | | | |
| PEG-70 mango glycerides | 1.0 | | | | |
| PEG-17 dimethicone | | 2.0 | | | |
| PEG-75 shea butter | | | 1.0 | | |
| glycerides | | | | | |
| Bis-PEG-18 methyl ether | | | | 3.0 | |
| dimethyl silane | | | | | |
| PEG-40 hydrogenated | | | | | 0.5 |
| castor oil | | | | | |
| Octadecenedioic acid | | 0.1 | | | |
| Salicylic acid | | 0.1 | | | |
| Ubiquinone | | 0.03 | | | |
| Creatine | | | | 1 | |
| Methylhydantoin-2-imide | | | | 0.1 | |
| Sodium hyaluronate | | | | | 0.1 |
| Ethanol | | | 10 | | |
| Na₂H₂EDTA | | 0.3 | | | |
| Perfume, preservative | q.s. | q.s. | q.s. | q.s. | q.s. |
| Dyes, etc. | q.s. | q.s. | q.s. | q.s. | q.s. |
| Sodium hydroxide | q.s. | q.s. | q.s. | q.s. | q.s. |
| Water | ad | ad | ad | ad | ad |
| | 100.0 | 100.0 | 100.0 | 100.0 | 100.0 |

1-10. (canceled)

11. A cosmetic preparation comprising:
a) hydroxyethyl acrylate/sodium acryloyldimethyltaurate copolymer, and
b) one or more water-soluble and/or water-dispersible lipids.

12. The cosmetic preparation of claim 11, wherein the cosmetic preparation comprises hydroxyethyl acrylate/sodium acryloyldimethyltaurate copolymer in a concentration of from 0.1 to 3.0% by weight, based on the total weight of the preparation.

13. The cosmetic preparation of claim 11, wherein the preparation comprises water-soluble and/or water-dispersible lipids in a total concentration of from 0.01 to 20.0% by weight, based on the total weight of the preparation.

14. The cosmetic preparation of claim 11, wherein one or more compounds selected from the group of compounds comprising ethoxylated (PEG or PPG) silicone oils (such as e.g. dimethicone or methyl ether dimethyl silicone), ethoxylated synthetic or natural esters or quaternized silicone oils are used as water-soluble and/or water-dispersible lipids.

15. The cosmetic preparation of claim 11, wherein the compound with the CAS No.: 111286-86-3 is used as the hydroxyethyl acrylate/sodium acryloyldimethyltaurate copolymer.

16. The cosmetic preparation of claim 11, wherein the preparation is in the form of a gel.

17. The cosmetic preparation of claim 16, wherein the gel is a transparent or translucent gel.

18. The cosmetic preparation of claim 11, wherein the preparation comprises one or more perfume substances selected from the group of compounds limonene [5989-27-5], citral, linalool [78-70-6], alpha-isomethylionone [1335-46-2], geraniol [106-24-1], citronellol [106-22-9], [24851-98-7], [18479-58-8], [54464-57-2], [80-54-6], [1222-05-5], [32388-55-9], [105-95-3], [31906-04-4], [8008-57-9], [32210-23-4], [120-57-0], [115-95-7], [101-86-0], [140-11-4], [6259-76-3] and [127-51-5], 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic acid diester, alpha-amylcinnamaldehyde, alpha-methylionone, amyl C, butylphenylmethylpropionalcinnamal, amyl salicylate, amylcinnamyl alcohol, anise alcohol, benzoin, benzyl alcohol, benzyl benzoate, benzyl cinnamate, benzyl salicylate, bergamot oil, bitter orange oil, butylphenylmethylpropional, cardamom oil, cedrol, cinnamal, cinnamyl alcohol, citronellyl methylcrotonate, lemon oil, coumarin, diethyl succinate, d-limonenes, ethyllinalool, eugenol, Evernia furfuracea extract, Evernia prunastri extract, farnesol, guaiac wood oil, hexylcinnamal, hexyl salicylate, hydroxycitronellal, hydroxyisohexyl 3-cyclohexenecarboxaldehyde, lavender oil, lemon oil, linalyl acetate, mandarin oil, menthyl PCA, methylheptenone, nutmeg oil, rosemary oil, sweet orange oil, terpineol, tonka bean oil, triethyl citrate and/or vanillin.

19. The cosmetic preparation of claim 11, wherein the preparation comprises one or more perfume substances in a total concentration of from 0.001 to 1% by weight, based on the total weight of the preparation.

20. The cosmetic preparation of claim 11, wherein the preparation comprises 0.1 to 15% by weight of glycerol, based on the total weight of the preparation.

21. The cosmetic preparation of claim 11, wherein the preparation comprises one or more solubility promoters in a total concentration of from 0.005 to 15% by weight, based on the total weight of the preparation.

22. The cosmetic preparation of claim 12, wherein the preparation comprises water-soluble and/or water-dispersible lipids in a total concentration of from 0.01 to 20.0% by weight, based on the total weight of the preparation.

23. The cosmetic preparation of claim 22, wherein one or more compounds selected from the group of compounds comprising ethoxylated (PEG or PPG) silicone oils (such as e.g. dimethicone or methyl ether dimethyl silicone), ethoxylated synthetic or natural esters or quaternized silicone oils are used as water-soluble and/or water-dispersible lipids.

24. The cosmetic preparation of claim 23, wherein the compound with the CAS No.: 111286-86-3 is used as the hydroxyethyl acrylate/sodium acryloyldimethyltaurate copolymer.

25. The cosmetic preparation of claim 24, wherein the preparation is in the form of a gel.

26. The cosmetic preparation of claim 25, wherein the gel is a transparent or translucent gel.

27. The cosmetic preparation of claim 25, wherein the preparation comprises one or more perfume substances selected from the group of compounds limonene [5989-27-5], citral, linalool [78-70-6], alpha-isomethylionone [1335-46-2], geraniol [106-24-1], citronellol [106-22-9], [24851-98-7], [18479-58-8], [54464-57-2], [80-54-6], [1222-05-5], [32388-55-9], [105-95-3], [31906-04-4], [8008-57-9], [32210-23-4], [120-57-0], [115-95-7], [101-86-0], [140-11-4], [6259-76-3] and [127-51-5], 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic acid diester, alpha-amylcinnamaldehyde, alpha-methylionone, amyl C, butylphenylmethylpropionalcinnamal, amyl salicylate, amylcinnamyl alcohol, anise alcohol, benzoin, benzyl alcohol, benzyl benzoate, benzyl cinnamate, benzyl salicylate, bergamot oil, bitter orange oil, butylphenylmethylpropional, cardamom oil, cedrol, cinnamal, cinnamyl alcohol, citronellyl methylcrotonate, lemon oil, coumarin, diethyl succinate, d-limonenes, ethyllinalool, eugenol, Evernia furfuracea extract, Evernia prunastri extract, farnesol, guaiac wood oil, hexylcinnamal, hexyl salicylate, hydroxycitronellal, hydroxyisohexyl 3-cyclohexenecarboxaldehyde, lavender oil, lemon oil, linalyl acetate, mandarin oil, menthyl PCA, methylheptenone, nutmeg oil, rosemary oil, sweet orange oil, terpineol, tonka bean oil, triethyl citrate and/or vanillin.

28. The cosmetic preparation of claim 27, wherein the preparation comprises one or more perfume substances in a total concentration of from 0.001 to 1% by weight, based on the total weight of the preparation.

29. The cosmetic preparation of claim 28, wherein the preparation comprises 0.1 to 15% by weight of glycerol, based on the total weight of the preparation.

30. The cosmetic preparation of claim 29, wherein the preparation comprises one or more solubility promoters in a total concentration of from 0.005 to 15% by weight, based on the total weight of the preparation.
